(19) United States
(12) Patent Application Publication
(10) Pub. No.: US 2022/0031916 A1
(43) Pub. Date: Feb. 3, 2022

(54) ZN-GA SERIES ALLOY AND ITS PREPARATION METHOD AND APPLICATION

(71) Applicant: University Of Science & Technology Beijing, Beijing (CN)

(72) Inventors: Huafang Li, Beijing (CN); Yixing Zheng, Shangrao City (CN); Luning Wang, Beijing (CN)

(21) Appl. No.: 17/141,609

(22) Filed: Jan. 5, 2021

(30) Foreign Application Priority Data
Aug. 3, 2020 (CN) 2020107654492

(51) Int. Cl. A61L 31/02; A61F 2/04; A61F 2/06; A61B 17/72; A61B 17/84; A61B 17/86; A61L 31/16; A61L 31/08; A61L 31/10; C22C 18/00; C22F 1/16

(52) U.S. Cl. CPC A61L 31/022; A61F 2002/2889; A61F 2/06; A61B 17/72; A61B 17/842; A61B 17/846; A61B 17/86; A61L 31/16; A61L 31/086; A61L 31/10; C22C 18/00; C22F 1/165; A61F 2002/047; A61F 2002/041; A61F 2002/046; A61F 2002/045; A61F 2002/044; A61F 2/04

(57) ABSTRACT

The invention discloses a Zn—Ga series alloy and a preparation method and application thereof, belonging to the technical field of medical alloys. The Zn—Ga series alloy includes Zn and Ga, and Ga accounts for 0-30 wt % but not including 0. The preparation method is to mix Zn and Ga or Zn, Ga and trace elements, then to obtain a Zn—Ga series alloy by coating paint after smelting or sintering. The mechanical properties of the prepared Zn—Ga series alloy meet the requirements of the strength and toughness of medical implant materials, and it can be degraded in vivo. It has the dual characteristics of biological corrosion degradation and suitable corrosion rate to provide long-term effective mechanical support.

### TECHNICAL FIELD

The invention relates to the technical field of medical alloys, in particular to a Zn—Ga series alloy and a preparation method and application thereof.

### BACKGROUND

The biomedical materials currently used in clinics mainly include biomedical metal materials, inorganic materials, polymer materials, composite materials and bionic materials. Compared with polymer materials and ceramic materials, medical metal materials have higher strength, toughness and processing properties, so they are the most widely used, such as 316L, 317L, 304V stainless steel, Co—Cr—Mo alloy, pure titanium, Ti-6A1-4V, TiNi alloy, and so on. These materials are non-degradable in the human body and are permanently implanted. After the service period of the implant in the human body expires, it must be removed through a second operation, which brings unnecessary physical pain and economic burden to the patient.

With the development of medicine and material science, for some materials that require temporary service, such as sutures, fracture fixation plates, vascular stents, biliary stents, etc., people hope that the material implanted in the body will only serve as a temporary replacement, and will gradually degrade and absorb with the regeneration of tissues or organs, so as to minimize the long-term impact of the material on the body. Because biodegradable materials are easy to interact with body fluids and other media in the body and gradually degrade, their decomposition products can be metabolized and eventually excreted from the body without the need for a second operation to remove them. Therefore, people pay more and more attention on it and it has become the current frontier and research hotspot in the field of international biomaterials.

The biodegradable materials commonly used in clinical practice are mainly biodegradable polymer materials and biodegradable ceramics. Although biodegradable polymer materials can be completely absorbed by the human body, their strength is low and it is difficult to provide structural support. The disadvantage of biodegradable ceramics is poor toughness and inability to coordinate deformation.

In recent years, biodegradable biomedical magnesium alloy materials have become one of the research hotspots. A series of biomedical biodegradable magnesium alloys have been developed, such as AZ31, WE43, Mg—Ca, etc. Although magnesium alloys have attractive application prospects as biomaterials, studies have found that magnesium alloys corrode too fast, and implants will quickly lose their mechanical integrity before tissues and organs are fully healed. Therefore, it is necessary to develop new degradable alloys to meet clinical needs.

Like magnesium and magnesium alloys, metallic zinc and its alloys are often used as anode materials sacrificed in corrosion protection due to their active chemical properties and easy corrosion. However, compared with magnesium, metallic zinc and its alloys have a higher corrosion potential. Therefore, compared with magnesium alloy, the corrosion rate of zinc and its alloy is slower, which is more in line with clinical needs, and is expected to be developed into new biomedical biodegradable implant materials and devices.

The normal zinc content in the human body is 2-3 grams. Zinc is the main component of dozens of enzymes in the body. Zinc is distributed in most organs and tissues, among which the content is higher in liver, muscle and bone. Although zinc is a trace element in the human body, it has a great effect. Known as the “spark plug of life”. (1) Zinc is related to various bone matrix synthase, and it can participate in bone formation and bone reconstruction. When zinc is deficient, the activity of a variety of zinc-containing enzymes in bone decreases, and bone growth is inhibited. (2) Zinc is a key component of biofilm, which plays an important role in maintaining the structure and function of more than 2,000 transcription factors and more than 300 enzymes. (3) Zinc can quickly enter endothelial cells and maintain the integrity of endothelial cells, reduce the susceptibility of blood vessels to atherosclerosis. (4) Zinc can protect cardiomyocytes from acute oxidative stress and inflammatory reactions caused by myocardial injury. (5) Zinc can actively participate in nucleic acid protein synthesis and accelerate wound healing; (6) In addition, zinc is also closely related to the metabolism of various cells in the body, such as sugar metabolism, lipid metabolism and anti-aging. Zinc deficiency can lead to arteriosclerosis, arrhythmia and failure, brain abnormalities, weakened immunity, diarrhea, loss of appetite, slowed growth, hair loss, night blindness, enlarged prostate, decreased male reproductive function, anemia, etc. Adults need to supplement 15-25 mg zinc daily.

Gallium (Ga) is a strong bone-fixing calcium agent for human body, which can be used to treat cancer-related hypercalcemia and osteitis deformans. Gallium has a strong bactericidal effect because it can bind to bacterial proteins. Gallium and its compounds have anti-inflammatory and anti-osteoporosis effects. Gallium can inhibit the resorption of osteoclasts, inhibit osteolysis, prevent the release of bone calcium, change the gene expression of type I collagen and fibrin in bone, can be beneficial to the formation of new bone, and can also increase the content of calcium and phosphorus in bone. It acts directly on the formation of human bones.

At present, there are no documents and patents at home and abroad that report the synthesis and performance of Zn—Ga series alloys, and no relevant documents and patents propose the use of Zn—Ga series alloys as degradable biomedical materials.

### SUMMARY OF THE INVENTION

The purpose of the present invention is to provide a Zn—Ga series zinc alloy and a preparation method and application thereof, in particular to a Zn—Ga series zinc alloy, a preparation method thereof, and application in the preparation of a body fluid-degradable medical implant. The zinc alloy prepared by the invention has excellent mechanical properties, can provide long-term effective support in the body, has excellent cell compatibility, blood compatibility, and tissue and organ compatibility, and can be used as biomedical implant materials.

In order to achieve the above objectives, the present invention provides the following solutions:

The present invention provides a Zn—Ga series alloy, including Zn and Ga, in which Ga accounts for 0-30 wt %, but not including 0.

As a further improvement of the present invention, the Zn—Ga series alloy also includes trace elements, which are magnesium, calcium, strontium, manganese, titanium, zirconium, germanium, copper, silicon, phosphorus, lithium, silver, tin and at least one of the rare earth elements. Wherein the trace element accounts for 0-10 wt %.

As a further improvement of the present invention, the surface of the Zn—Ga series alloy is further coated with a degradable high polymer coating, a degradable ceramic coating or a degradable drug coating.

As a further improvement of the present invention, the thickness of the degradable polymer coating, the degradable ceramic coating and the degradable drug coating are all 0.001 to 5 mm.

As a further improvement of the present invention, the preparation material of the degradable polymer coating is at least one of the following 1) and 2).

1) Any one of polycaprolactone, polylactic acid, polyglycolic acid, L-polylactic acid, polycyanoacrylate, polyanhydride, polyphosphazene, polydioxanone, polyhydroxybutyrate and polyhydroxyvalerates;

2) A copolymer of any two or more of polylactic acid, polycaprolactone, polyglycolic acid, L-polylactic acid, polycyanoacrylate, and polydioxanone;

The preparation material of the degradable ceramic coating is at least one of hydroxyapatite, tricalcium phosphate or tetracalcium oxyphosphate;

The degradable drug coating is at least one of rapamycin and its derivatives coating, paclitaxel coating, everolimus coating, sirolimus coating, mitomycin coating and antibacterial coating One kind.

As a further improvement of the present invention, the Zn—Ga series zinc alloy is specifically any one of the following 1)-4), in a mass percentage,

1) composed of 95˜99% Zn and 1%˜5% Ga;

2) composed of 99% Zn and 1% Ga;

3) composed of 98% Zn and 2% Ga;

4) composed of 98.5% Zn, 1% Ga and 0.5% Y.

The Zn—Ga series zinc alloy prepared by the invention has a dense structure or a porous structure, has good tissue compatibility, and is a reliable biomedical implant material.

The present invention also provides a method for preparing the Zn—Ga series alloy, which includes the following steps:

Mix Zn, Ga and the trace elements according to any one of the following methods 1) and 2) to obtain a mixture.

1) Zn and Ga;

2) Zn, Ga and trace elements;

The zinc alloy can be obtained according to the following steps a) or b).

a) Under the protection of CO₂ and SF₆ atmosphere, the mixture is smelted or sintered, and the zinc alloy is obtained after cooling;

b) Under the protection of CO₂ and SF₆ atmosphere, the mixture is smelted or sintered, and the degradable polymer coating, the degradable ceramic coating or the degradable drug coating is coated after cooling to obtain the zinc alloy. The method of preparing the zinc alloy also includes the step of applying a coating is to meet different clinical needs.

As a further improvement of the present invention, the melting temperature in the preparation method is 500 to 700° C.

As a further improvement of the present invention, the preparation method further includes machining steps of zinc alloy

As a further improvement of the present invention, the mechanical processing is at least one of rolling, forging, rapid solidification and extrusion.

As a further improvement of the present invention, repeated rolling is performed in a back rolling mill, the hot rolling temperature is 250° C., and finally in a finishing rolling mill, it is rolled to a thickness of 1.5 mm at 250° C.

As a further improvement of the present invention, the forging includes the steps of heat-retaining the Zn—Ga series alloy at a temperature of 150-200° C. and forging at a temperature of 200-300° C., and the heat-retaining time is 3˜50 h, the forging speed rate is not less than 350 mm/s.

As a further improvement of the present invention, the extrusion temperature is 150-250° C., specifically 200-220° C.; and the extrusion ratio is 10-70, specifically 20-25.

As a further improvement of the present invention, the rapid solidification includes the following steps—

under the protection of Ar gas, a high vacuum rapid quenching system is used to prepare a rapid solidification thin strip, and then the thin strip is crushed into powder. Then under the condition of 200˜350° C., carry out vacuum hot pressing for 1˜24 h.

As a further improvement of the present invention, the settings of the high-vacuum rapid quenching system are as follows:

the feeding amount is 2-8 g, the induction heating power is 3-7 kW, the distance between the nozzle and the roller is 0.80 mm, the spray pressure is 0.05-0.2 MPa, and the roller speed is 500-3000 r/min and the nozzle slit size is 1 film×8 mm×6 mm.

As a further improvement of the present invention, the sintering is any one of the following methods element powder mixed sintering method, pre-alloyed powder sintering method, and self-propagating high-temperature synthesis method.

As a further improvement of the present invention, the element powder mixing and sintering method is to uniformly mix the raw materials for preparing the porous structure Zn—Ga series alloy, press it into a green body, and then in a vacuum sintering furnace, slowly heat up to 100-200° C. at 2˜4° C./min, then quickly heat up to 200˜300° C. at 30° C./min for sintering, then lower the temperature to obtain a porous structure of Zn—Ga series alloy.

As a further improvement of the present invention, the pre-alloyed powder sintering method is to mix the raw materials for preparing the porous structure Zn—Ga series alloy and then perform high-energy ball milling, then press molding, and perform heat treatment at 250-350° C. for 10-20 hours to obtain a porous structure of Zn—Ga series alloy;

As a further improvement of the present invention, the self-propagating high-temperature synthesis method is to mix the raw materials for preparing porous structure Zn—Ga series alloys and press them into billets, under the protection of inert gas, with the pressure 1×10³1×10⁵ Pa and temperature 250˜350° C. Then the Zn—Ga series alloy blank is ignited for self-propagating high-temperature synthesis to obtain a Zn—Ga series alloy with porous structure.

As a further improvement of the present invention, the method for coating the biodegradable polymer coating is to take the zinc alloy for acid pickling. Then it is immersed in the preparation material of the biodegradable polymer coating in a colloid prepared by trichloroethane for 10 to 30 minutes, and then pulling out at a uniform speed for centrifugal treatment to obtain a zinc alloy with biodegradable polymer coating.

As a further improvement of the present invention, the method for applying the degradable ceramic coating can be any one of plasma spraying, electrophoretic deposition, anodizing and hydrothermal synthesis;

As a further improvement of the present invention, the main plasma gas used in plasma spraying is Ar, the flow rate is 30-100 scfh, the secondary plasma gas is H₂, the flow rate is 5-20 scfh, and the spraying current is 400-800 A. The spraying voltage is 40˜80V, and the spraying distance is 100-500 mm.

As a further improvement of the present invention, the method for electrodeposition of the degradable ceramic coating is to use a zinc alloy as a cathode in an electrolyte containing calcium and phosphorus salts with a current density of 2-10 mA/cm², and after treatment for 10-60 min, washing and drying to obtain the surface modified zinc alloy;

As a further improvement of the present invention, the method of combining anodization and hydrothermal synthesis is to oxidize the zinc alloy in an electrolyte containing 0.01 to 0.5 mol/L β-glycerophosphate sodium and 0.1 to 2 mol/L calcium acetate, at 200˜500V for 10˜30 min, and then treat the zinc alloy at 200˜400° C. for 1˜4 h.

As a further improvement of the present invention, the method for applying the degradable drug coating is a physical and chemical method;

The physical method coating process mainly uses immersion and spraying methods; the chemical method mainly uses electrochemical principles for electroplating;

The soaking method is to prepare a solution of the active drug and a controlled release carrier (or a separate active drug), and the specific concentration may vary due to the viscosity of the solution and the required drug dosage. Then, the medical implant is immersed in the solution, and then undergoes necessary post-treatment processes, such as cross-linking, drying, curing, etc., to form a drug coating;

The spraying method is to prepare a solution of the active drug and a controlled release carrier (or a separate active drug), and then uniformly coat the solution on the surface of the medical implant through a spraying tool or a special spraying device, After drying, curing and other post-processing steps, the drug coating is made;

The chemical method uses active drugs and (or) a controlled release carrier to generate an electrical oxidation-reduction reaction on the electrode made by the medical implant, so that the medical implant surface forms a stable drug coating connected by chemical bonds.

According to the characteristics of Zn and Zn alloy easy to corrode, the invention selects Zn—Ga alloy as degradable material for medical implants. The mechanical properties of the Zn—Ga series alloy of the present invention meet the requirements of the strength and toughness of medical implant materials, and at the same time it can be degraded in vivo, that is, it can overcome the low strength of medical polymer materials and the indegradability of traditional medical metal materials such as 316L stainless steel, titanium and titanium alloys. It can also overcome the defect that the excessive degradation rate of magnesium and magnesium alloy leads to the loss of mechanical properties of the implant. It has the dual characteristics of “bio-corrosive degradation characteristics” and “appropriate corrosion rate to ensure long-term effective mechanical support”.

The present invention also provides the application of the Zn—Ga series alloy, which is used to prepare a body fluid-degradable medical implant which includes a therapeutic implanted stent, a bone repair instrument, and a cranio-maxillofacial repair instrument.

As a further improvement of the present invention, the therapeutic implantable stent may be a blood vessel stent, an esophageal stent, an intestinal stent, a tracheal stent, a biliary stent or a urethral stent.

The bone repair instrument can be a bone tissue repair bracket, a bone connector, a fixation wire, a fixation screw, a fixation rivet, a fixation pin, a bone splint, an intramedullary nail or a bone sleeve.

The cranio-maxillofacial repair instrument can be a cranial bone repair net, a maxillofacial bone defect repair bracket, etc.

The present invention discloses the following technical effects:

(1) The mechanical properties of the Zn—Ga series alloy prepared by the present invention meet the requirements of the strength and toughness of medical implant materials, and at the same time it is degradable in vivo. It has the dual characteristics of “bio-corrosive degradation characteristics” and “appropriate corrosion rate to ensure long-term effective mechanical support”.

(2) When the Zn—Ga series alloy of the present invention is used in a degradable medical implant, it can not only exert the high strength characteristics of its metal material within a period of implantation to complete the function of the implant (such as inducing the formation of new bone tissue or supporting narrow blood vessels), and it can be gradually corroded and degraded by the human body as a “foreign body” while the diseased part of the human body repairs itself. The quantity and volume are gradually reduced, and the dissolved metal ions can be absorbed and utilized by the organism to promote bone growth or metabolism to be eliminated from the body, and finally the metal material implant completely degrades and disappears when the body finishes its self-repair.

(3) The body fluid-degradable medical implant provided by the present invention is non-toxic and has good tissue compatibility and blood compatibility.

### BRIEF DESCRIPTION OF THE FIGURES

In order to explain the embodiments of the present invention or the technical solutions in the prior art more clearly, the following will briefly introduce the drawings that need to be used in the embodiments. Obviously, the drawings in the following description are only some embodiments of the present invention. For those of ordinary skill in the art, other drawings can be obtained based on these drawings without creative labor.

FIG. 1 shows the cell compatibility test results of Zn—Ga alloy.

### DETAILED DESCRIPTION OF THE INVENTION

Various exemplary embodiments of the present invention will now be described in detail. The detailed description should not be considered as a limitation to the present invention, but should be understood as a more detailed description of certain aspects, characteristics, and embodiments of the present invention.

It should be understood that the terms described in the present invention are only used to describe specific embodiments and are not used to limit the present invention. In addition, for the numerical range in the present invention, it should be understood that each intermediate value between the upper limit and the lower limit of the range is also specifically disclosed. Each smaller range between any stated value or intermediate value within the stated range and any other stated value or intermediate value within the stated range is also included in the present invention. The upper and lower limits of these smaller ranges can be independently included or excluded from the range.

Unless otherwise specified, all technical and scientific terms used herein have the same meaning as commonly understood by those skilled in the art in the field of the present invention. Although the present invention only describes preferred methods and materials, any methods and materials similar or equivalent to those described herein can also be used in the practice or testing of the present invention. All documents mentioned in this specification are incorporated by reference to disclose and describe methods and/or materials related to the documents. In the event of conflict with any incorporated document, the content of this manual shall prevail.

Without departing from the scope or spirit of the present invention, various improvements and changes can be made to the specific embodiments of the present specification, which is obvious to those skilled in the art. Other embodiments derived from the description of the present invention will also be obvious to the skilled person. The specification and examples of this application are only exemplary.

As used herein, “including”, “included”, “having”, “containing”, etc., are all open terms, which means including but not limited to.

The percentages used in the following examples are all mass percentages unless otherwise specified.

### Example 1. Preparation of as-Cast Zn—Ga Series Alloy

Using pure Zn (99.99 wt. %) and pure Ga (99.95 wt. %) (purchased from Beijing Cuibailin Nonferrous Metal Technology Development Center) as raw materials. Mixing according to different mass ratios—the mass ratio of Zn to Ga and other trace elements Y(Gd, Nd) is 99:1, 98.5:1.5, 98:2, 97:3, 95:5) and smelting at 550° C. under the protection of CO₂+SF₆ atmosphere. After the raw materials are fully melted, holding for 10 minutes, then using circulating water to cool quickly and the Zn—Ga alloy ingot was prepared.

### Example 2. Preparation of Rolled Zn—Ga Series Alloy

First, prepare as-cast Zn—Ga alloy ingots according to the steps in Example 1. Then, the obtained Zn—Ga alloy ingots were hot rolled, and the ingots were preheated at 250° C. Then, it is rolled repeatedly in a reciprocating mill by hot rolling at a warm rolling temperature of 250° C. and finally rolled to a thickness of 1.5 mm in a finishing mill at 250° C.

### Example 3. Preparation of Extruded Zn—Ga Series Alloy

Follow the steps in 1) or 2) below to prepare.

1) First, prepare as-cast Zn—Ga series alloy ingots according to the steps in Example 1 and prepare Zn—Ga series alloy bars by extrusion. Using radial extrusion with an extrusion temperature 200° C. The extrusion ratio is 20. Then a Zn—Ga series alloy bar with a diameter of 10 mm was prepared.

2) First, prepare as-cast Zn—Ga series alloy ingots according to the steps in Example 1, and use high vacuum rapid quenching system to prepare rapidly solidified Zn—Ga series alloy thin strips. The specific method is that the raw materials are mixed according to the stated ratio and then a high vacuum rapid quenching system is used to prepare a rapidly solidified Zn—Ga ribbon (temperature 550° C., no hot pressing time). The parameters are feeding amount 2˜8 g, induction heating power 3˜7 kW, nozzle and roller distance 0.80 mm, spray pressure 0.1 MPa, roller speed 2000 r/min and nozzle slit size 1 film×8 mm×6 mm. Then the thin strip is crushed and pressed into a billet. The Zn—Ga series alloy bar is prepared by extrusion. Using radial extrusion with an extrusion temperature 200° C. and an extrusion ratio 20, and then the Zn—Ga series alloy bars with a diameter of 10 mm is prepared.

### Example 4. Zn—Ga Series Alloy Mechanical Properties

The Zn—Ga series alloys, prepared according to the methods of Examples 1-3, were respectively prepared into tensile samples according to ASTM-E8-04 tensile test standard, and then polished by 400#, 800#, 1200# and 2000# SiC sandpaper series. After ultrasonic cleaning in acetone, absolute ethanol and deionized water for 15 minutes, a universal material mechanics testing machine was used to perform a tensile test at room temperature, and the tensile speed was 1 mm/min.

The tensile properties at room temperature of each sample of the Zn—Ga series alloy are shown in Table 1. From Table 1, it can be seen that the yield strength and tensile strength of the rolled alloy and the extruded alloy are obviously improved compared to the as-cast alloy. At the same time, the elongation has been greatly increased, indicating that the mechanical properties of the material have been further optimized after the deformation process.

TABLE 1
Zn—Ga alloy tensile mechanical properties data

| | Tensile | Yield | |
|---|---|---|---|
| Sample No | strength/MPa | strength/MPa | Elongation/% |
| Pure zinc ingot | 22.32 | 13.53 | 0.25 |
| Zn—5Ga ingot | 103.38 | 78.06 | 1.15 |
| Zn—1Ga—0.5Y | 269.21 | 210.05 | 26.91 |
| rolled plate | | | |
| Zn—1Ga—0.5Y | 290.39 | 243.60 | 18.05 |
| bar | | | |
| Zn—1Ga—0.5Y | 183.39 | 135.26 | 2.57 |
| casting ingot | | | |

### Example 5. Zn—Ga Alloy Blood Compatibility

The rolled Zn—Ga alloy of Example 2 was prepared into a 10×10×1.5 mm Zn—Ga alloy sample piece by wire cutting, which was polished by 400#, 800#, 1200# and 2000# SiC sandpaper series. After ultrasonic cleaning for 15 minutes in acetone, absolute ethanol and deionized water, they were dried at 25° C. Fresh blood from healthy volunteers was collected and stored in an anticoagulant tube containing 3.8 wt % sodium citrate as an anticoagulant. Dilute with 0.9% normal saline at a ratio of 4:5 to prepare a diluted blood sample. Soak the sample in 10 mL of normal saline, keep it at 37±0.5° C. for 30 min, add 0.2 mL of diluted blood sample, and keep it at 37±0.5° C. for 60 min. 10 mL of normal saline was used as the negative control group, and 10 mL of deionized water was used as the positive control group. After centrifugation at 3000 rpm for 5 minutes, the supernatant was taken to measure the absorbance OD value with an Unic-7200 UV-Vis spectrophotometer at 545 nm, and three sets of parallel samples were set for statistical analysis.

Use the following formula to calculate the hemolysis rate:

Hemolysis rate=(experimental group OD value-negative group OD value)/(positive group OD value-negative group OD value)×100%.

The experimental results show that the hemolysis rate of Zn—Ga alloy is between 0.2% and 0.5%, which is far less than the safety threshold of 5% required for clinical use, and shows good compatibility of red blood cells and hemoglobin.

### Example 7. Preparation of Body Fluid Degradable Medical Zn—Ga Implant and its Cell Compatibility Experiment

The Zn—Ga alloy was prepared according to the method of Examples 1-3. The 6 Zn—Ga alloy blocks prepared above with length, width, and thickness of 10 mm, 10 mm, and 1.5 mm respectively were sterilized by γ-ray and placed in a sterile culture flask. Add MEM cell culture medium at the ratio of sample surface area to MEM cell culture medium volume of 1.25 cm²/mL, and place it in an incubator at 37° C., 95% relative humidity, and 5% CO₂ for 72 hours to obtain Zn—Ga alloy extraction liquid stock solution. Seal it and keep it in refrigerator at 4° C. for later use.

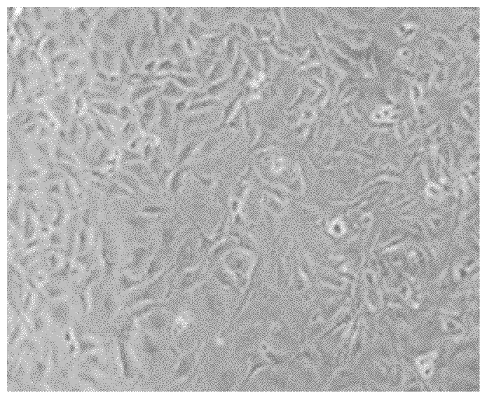

Extraction and cell inoculation culture and observation result. MG63 cells (purchased from Guangzhou Genio Biotechnology Co., Ltd.) were resuscitated and passaged, suspended in MEM cell culture medium, and inoculated on 96-well culture plates. The negative control group was added with MEM cell culture medium, and the Zn—Ga alloy extract group was added with the 4-fold diluted Zn—Ga alloy extract obtained above, so that the final cell concentration was 5×10⁴/mL. Culture in a 37° C., 5% CO₂ incubator. After 5 days, take out the culture plate and observe the morphology of living cells under an inverted phase contrast microscope (as shown in FIG. 1). The results showed that the cell morphology showed healthy and stretched spindle-shaped convergent growth, indicating that Zn—Ga alloy has excellent cell compatibility.

The above-mentioned embodiments only describe the preferred modes of the present invention, and do not limit the scope of the present invention. Without departing from the design spirit of the present invention, various modifications and improvements made by those of ordinary skill in the art to the technical solution of the present invention shall fall within the protection scope determined by the claims of the present invention.

1. A Zn—Ga series alloy is characterized in that it comprises Zn and Ga, and Ga accounts for 0-30 wt %, but not including 0.

2. The Zn—Ga series alloy according to claim 1 is characterized in that the Zn—Ga series alloy further includes trace elements, which is at least one of magnesium, calcium, strontium, manganese, titanium, zirconium, germanium, copper, silicon, phosphorus, lithium, silver, tin and rare earth elements.

3. The Zn—Ga series alloy according to claim 2 is characterized in that the trace element accounts for 0-10 wt %.

4. The Zn—Ga series alloy according to claim 1 is characterized in that the surface of the Zn—Ga series alloy is further coated with a degradable polymer coating, a degradable ceramic coating or a degradable drug coating.

5. The Zn—Ga series alloy according to claim 1 is characterized in that the preparation material of the degradable polymer coating is at least one of the following 1) and 2):
1) any one of polycaprolactone, polylactic acid, polyglycolic acid, L-polylactic acid, polycyanoacrylate, polyanhydride, polyphosphazene, polydioxanone, polyhydroxybutyrate and polyhydroxyvalerates;
2) a copolymer of any two or more of polylactic acid, polycaprolactone, polyglycolic acid, L-polylactic acid, polycyanoacrylate, and polydioxanone;
the preparation material of the ceramic coating is at least one of hydroxyapatite, tricalcium phosphate or tetracalcium oxyphosphate;
the drug coating is at least one of rapamycin and its derivative coatings, paclitaxel coatings, everolimus coatings, sirolimus coatings, mitomycin coatings and antibacterial coatings.

6. A method for preparing a Zn—Ga series alloy according to claim 1 is characterized in that it comprises the following steps:
Mix Zn, Ga and the trace elements according to any one of the following methods 1) and 2) to obtain a mixture.
1) Zn and Ga;
2) Zn, Ga and trace elements;
the zinc alloy can be obtained according to the following steps a) or b).
a) under the protection of CO2 and SF6 atmosphere, the mixture is smelted or sintered, and the zinc alloy is obtained after cooling;
b) under the protection of CO2 and SF6 atmosphere, the mixture is smelted or sintered, and the degradable polymer coating, the degradable ceramic coating or the degradable drug coating is coated after cooling to obtain the Zn—Ga series alloy.

7. The method for preparing the Zn—Ga series alloy according to claim 6 is characterized in that it further comprises a step of machining the Zn GA series alloy.

8. The method for preparing a Zn—Ga series alloy according to claim 7 is characterized in that the mechanical processing is at least one of rolling, forging, rapid solidification and extrusion.

9. The method for preparing a Zn—Ga series alloy according to claim 6 is characterized in that the sintering is any one of the following methods—element powder mixed sintering method, pre-alloyed powder sintering method, and self-propagating high-temperature synthesis method.

10. An application of the Zn—Ga series alloy according to claim 1 is characterized in that the Zn—Ga series alloy is used in preparing a body fluid-degradable medical implant.
